SEQUENCE LISTING (47 sequences)

SEQ ID NO: 1 - protein, 79 aa, Homo sapiens
SEAEDASLLSFMQGYMKHATKTAKDALSSVQESQVAQQARGWVTDGFSSLKDYWSTVKDKFSEFWDLDPEVRPTSAVAA

SEQ ID NO: 2 - protein, 24 aa, Homo sapiens
MQGYMKHATKTAKDALSSVQESQV

SEQ ID NO: 3 - protein, 10 aa, Homo sapiens
MQGYMKHATK

SEQ ID NO: 4 - protein, 32 aa, Homo sapiens
DGFSSLKDYWSTVKDKFSEFWDLDPEVRPTSA

SEQ ID NO: 5 - protein, 21 aa, Homo sapiens
DGFSSLKDYWSTVKDKFSEFW

SEQ ID NO: 6 - protein, 8 aa, Homo sapiens
MQGYMKHA

SEQ ID NO: 7 - protein, 8 aa, Homo sapiens
QGYMKHAT

SEQ ID NO: 8 - protein, 8 aa, Homo sapiens
GYMKHATK

SEQ ID NO: 9 - protein, 8 aa, Homo sapiens
YMKHATKT

SEQ ID NO: 10 - protein, 8 aa, Homo sapiens
MKHATKTA

SEQ ID NO: 11 - protein, 8 aa, Homo sapiens
KHATKTAK

SEQ ID NO: 12 - protein, 8 aa, Homo sapiens
HATKTAKD

SEQ ID NO: 13 - protein, 8 aa, Homo sapiens
ATKTAKDA

SEQ ID NO: 14 - protein, 8 aa, Homo sapiens
TKTAKDAL

SEQ ID NO: 15 - protein, 8 aa, Homo sapiens
KTAKDALS

SEQ ID NO: 16 - protein, 8 aa, Homo sapiens
TAKDALSS

SEQ ID NO: 17 - protein, 8 aa, Homo sapiens
AKDALSSV

SEQ ID NO: 18 - protein, 8 aa, Homo sapiens
KDALSSVQ

SEQ ID NO: 19 - protein, 8 aa, Homo sapiens
DALSSVQE

SEQ ID NO: 20 - protein, 8 aa, Homo sapiens
ALSSVQES

SEQ ID NO: 21 - protein, 8 aa, Homo sapiens
LSSVQESQ

SEQ ID NO: 22 - protein, 8 aa, Homo sapiens
SSVQESQV

SEQ ID NO: 23 - protein, 8 aa, Homo sapiens
DGFSSLKD

SEQ ID NO: 24 - protein, 8 aa, Homo sapiens
GFSSLKDY

SEQ ID NO: 25 - protein, 8 aa, Homo sapiens
FSSLKDYW

SEQ ID NO: 26 - protein, 8 aa, Homo sapiens
SSLKDYWS

SEQ ID NO: 27 - protein, 8 aa, Homo sapiens
SLKDYWST

SEQ ID NO: 28 - protein, 8 aa, Homo sapiens
LKDYWSTV

SEQ ID NO: 29 - protein, 8 aa, Homo sapiens
KDYWSTVK

SEQ ID NO: 30 - protein, 8 aa, Homo sapiens
DYWSTVKD

SEQ ID NO: 31 - protein, 8 aa, Homo sapiens
YWSTVKDK

SEQ ID NO: 32 - protein, 8 aa, Homo sapiens
WSTVKDKF

SEQ ID NO: 33 - protein, 8 aa, Homo sapiens
STVKDKFS

SEQ ID NO: 34 - protein, 8 aa, Homo sapiens
TVKDKFSE

SEQ ID NO: 35 - protein, 8 aa, Homo sapiens
VKDKFSEF

SEQ ID NO: 36 - protein, 8 aa, Homo sapiens
KDKFSEFW

SEQ ID NO: 37 - protein, 8 aa, Homo sapiens
DKFSEFWD

SEQ ID NO: 38 - protein, 8 aa, Homo sapiens
KFSEFWDL

SEQ ID NO: 39 - protein, 8 aa, Homo sapiens
FSEFWDLD

SEQ ID NO: 40 - protein, 8 aa, Homo sapiens
SEFWDLDP

SEQ ID NO: 41 - protein, 8 aa, Homo sapiens
EFWDLDPE

SEQ ID NO: 42 - protein, 8 aa, Homo sapiens
FWDLDPEV

SEQ ID NO: 43 - protein, 8 aa, Homo sapiens
WDLDPEVR

SEQ ID NO: 44 - protein, 8 aa, Homo sapiens
DLDPEVRP

SEQ ID NO: 45 - protein, 8 aa, Homo sapiens
LDPEVRPT

SEQ ID NO: 46 - protein, 8 aa, Homo sapiens
DPEVRPTS

SEQ ID NO: 47 - protein, 8 aa, Homo sapiens
PEVRPTSA

(19) United States
(12) Patent Application Publication
(10) Pub. No.: US 2004/0052809 A1
(43) Pub. Date: Mar. 18, 2004

(54) Vaccine

(76) Inventors: Pascal Mettens, Rixensart (BE); Philippe Monteyne, Rixensart (BE)

Correspondence Address: GLAXOSMITHKLINE, Corporate Intellectual Property - UW2220, P.O. Box 1539, King of Prussia, PA, 19406-0939, US

(21) Appl. No.: 10/387,957

(22) Filed: Aug. 20, 2003

(30) Foreign Application Priority Data
Aug. 29, 2002 (WO) PCT/EP02/09650

(51) Int. Cl. A61K 39/08; C07K 14/195; C07K 14/705

(52) U.S. Cl. 424/185.1; 530/350; 424/239.1; 530/403

(57) ABSTRACT

The present invention relates to novel vaccine therapies, and prophylactic treatments of atherosclerotic diseases. Accordingly there is provided, immunogens comprising specific fragments or derivatives of Apolipoprotein C-III (ApoCIII). The vaccines of the present invention, comprising said immunogens, are potent in the prevention, or reduction, of atherosclerotic plaque formation over prolonged periods of time, thereby reducing the potential of atherosclerosis leading to coronary or cerebrovascular disease. Also provided are methods of treating or preventing atherosclerosis by active vaccination, or passive vaccination through administration to a patient of an antibody that is capable of binding to the specific fragments of ApoCIII. Specific monoclonal antibodies and their use in therapy of atherosclerosis is provided. There is further provided the use of the immunogens of the present invention in medicine, and methods of their production. The fragments of ApoCIII which form the basis of the immunogens of the present invention, and also the targets for passive immunotherapy, are encompassed within the regions between amino acid numbers 45-76 and, particularly, 12-35 of the mature form of human ApoCIII.

The present invention relates to novel vaccine therapies, and prophylactic treatments of atherosclerotic diseases. Accordingly there is provided, immunogens capable of inducing an immune response against specific epitopes of Apolipoprotein C-III (ApoCIII). The vaccines of the present invention, comprising said immunogens, are potent in the prevention, or reduction, of atherosclerotic plaque formation over prolonged periods of time, thereby reducing the potential of atherosclerosis leading to coronary or cerebrovascular disease. Also provided are methods of treating or preventing atherosclerosis by passive vaccination through administration to a patient of an antibody that is capable of binding to the specific fragments of ApoCIII. Specific monoclonal antibodies and their use in therapy of atherosclerosis are provided. There is further provided the use of the immunogens of the present invention in medicine, and methods of their production. The epitopes of ApoCIII which form the basis of the immunogens of the present invention, and also consist of the targets for the passive immunotherapy aspects of the present invention, are encompassed within the regions between amino acid numbers 12-35 and between amino acid numbers 45-76 (particularly 45-65) of the mature form of human ApoCIII.

Atherosclerosis is the leading cause of death and disability in the developed world, and is the major cause of coronary and cerebrovascular deaths, with approximately 7.2 and 4.6 million deaths per year worldwide respectively (Atherosclerosis is generally described in Harrison's Principles of Internal Medicine (14^{th} Edition, McGraw Hill, p1345-1352), Berliner, J. et al., 1995, Circulation, 91:2488-2496; Ross, R., 1993; Nature, 362:801). The name in Greek refers to the thickening (sclerosis) of the arterial intima and accumulation of lipid (athere) in lesions.

Although many generalised or systemic risk factors predispose to its development, such as a high cholesterol diet and smoking, this disease may affect different distinct regions of the circulation. For example, atherosclerosis of the coronary arteries commonly causes angina pectoris and myocardial infarction. Whilst, atherosclerosis of the arteries supplying the central nervous system frequently provokes transient cerebral ischemia and strokes. In the peripheral circulation, atherosclerosis can cause intermittent claudication and gangrene and can jeopardise limb viability. Involvement of the splanchnic circulation can cause mesenteric ischemia and bowel infarction. Atherosclerosis can affect the kidney directly (eg causing renal artery stenosis), and in addition, the kidney is a frequent site of atheroembolic disease.

Atherogenesis in humans typically occurs over many years, usually many decades. The slow build up of atherogenic plaques in the lining of the vasculature can lead to chronic clinical expressions through blood flow restriction (such as stable effort-induced angina pectoris or predictable and reproducible intermittent claudication). Alternatively, a much more dramatic acute clinical event, such as a myocardial infarction or cerebrovascular accident can occur after plaque rupture. The way in which atherosclerosis affects an arterial segment also varies, an additional feature of the heterogeneity and complexity of this disease. Atheromas are usually thought of as stenotic lesions, or plaques, which can limit blood flow, however, atherosclerosis can also cause ectasia and development of aneurysmal disease with an increase in lumen caliber. This expression of atherosclerosis frequently occurs in the aorta, creating a predisposition to rupture or dissection rather than to stenosis or occlusion.

The genesis of atherogenic plaques has been studied in depth. In normal human adults, the intimal layer of arteries contains some resident smooth muscle cells embedded in extracellular matrix and is covered with a monolayer of vascular endothelial cells. Initial stages of atherogenesis involve the development of “fatty streaks” in the walls of the blood vessel resulting from accumulation and deposit of lipoproteins in regions of the intimal layer of the artery. Low-density lipoprotein (LDL) particles, rich in cholesterol, is an example of an atherogenic lipoprotein which is capable of deposition in the vessel walls to form such fatty streaks.

Once deposited within the vessel wall, the lipoprotein particles undergo chemical modification, including both oxidation and non-enzymatic glycation. These oxidised and glycated lipoproteins then contribute to many of the subsequent events of lesion development. The chemical modifications attract macrophages within the vessel walls, which internalise the oxidised LDL and become foam cells which initiate lesions called plaques. It is the atherosclerotic plaques which are responsible for the clinical manifestations of atherosclerosis, either they limit blood flow, or allow aneurism, or may even rupture provoking the coronary or cerebrovascular attacks.

The development of atherosclerosis is a long process which may occur over decades, which is initiated by an imbalance between atherogenic and protective lipoproteins. For example, cholesterol associated with high-density lipoproteins or HDL (so called “good” cholesterol) and low-density lipoproteins or LDL (so called; “bad” cholesterol) levels in the circulation are thought to be markers of increased probability of atherosclerosis (Harrison's Principles of Internal Medicine (14^{th} Edition, McGraw Hill, p1345-1352)).

Cholesterol, cholesterol esters, triacylglycerols and other lipids are transported in body fluids by a series of lipoproteins classified according to their increasing density: chylomicrons, Very Low, Low, Intermediate and High density lipoproteins (CM, VLDL, LDL, IDL and HDL respectively). These lipoprotein-complexes consist of a core of hydrophobic lipids surrounded by polar lipids and then by a shell of Apolipoproteins. Currently, there are at least twelve types of apolipoproteins known, A-I, A-II, A-IV, A-V, B, CI, CII, CIII, D, E, H and J. There are at least two functions of these apolipoproteins which are common to all lipoprotein complexes, first they are responsible for the solubilisation of the hydrophobic lipid cores that they carry, and second they are also involved in the regulation of cholesterol lipoprotein uptake by specific cells. The different types of lipoproteins may have different functions, for example LDL (which are rich in cholesterol esters) are thought to be associated with the transport of cholesterol to peripheral tissues for new membrane synthesis.

One of these apolipoproteins, apolipoprotein C-III (ApoCIII), is a 79 amino acid protein produced in the liver and intestine (Brewer et al., J. Biol. Chem. (1974), 249: 4975-4984; Protter, A. A., et al., 1984, DNA, 3:449-456; Fruchart, J. C. et al, 1996, Drugs Affecting Lipid Metabolism, (Eds. Gotto, A. M. et al.), Kluwer Academic Publishers and Fordazione Giovanni Lorenzini, Netherlands, p631-638; Claveny, V. et al., Arteriosclerosis, Thrombosis and Vascular Biology, 15, 7, 963-971; U.S. Pat. No. 4,801,531; McConathy, W. J. et al. 1992, Journal of Lipid Research, 33, 995-1003). Apo CIII is a component of CM, VLDL, LDL (Lenich et al., C., J. Lip. Res. (1988) 29, 755-764), and also HDL, and exists as three isoforms: apo CIII0, apo CIII1 and apo CIII2. Apo CIII0 is not glycosylated, however apo CIII1 and apo CIII2 are glycosylated and have respectively one and two sialic acid residues (Ito et al., 1989 J. lipd. Res. November 30:11 1781-1787). The sugar moiety consists of disaccharide β-D galactosyl (1-3) α-N-Acetyl-D-Galactosamine attached to threonine 74 of protein chain by O-glycosidic binding (Assman et al., 1989, BBA 541:234-240). In human normolipidemic plasma apo CIII0, apo CIII1 and apo CIII2 represent 14%, 59% and 27% of total apo CIII respectively. Mutagenesis of the glycosylation site of human apo CIII doesn't affect its secretion and lipid binding (Roghani et al., 1988 JBC 34:17925-32).

Mature Human ApoCIII has the following amino acid sequence:

₁SEAEDASLLSFMQGYMKHATKTAKDALSSVQESQVAQQARGWVTDGFSS LKDYWSTVKDKFSEFWDLDPEVRPTSAVAA₇₉ (SEQ ID. NO. 1).

Plasma concentration of apo CIII is positively correlated with levels of plasma triglycerides (Schonfeld et al., Metabolism (1979) 28: 1001-1010; Kaslyap et al., J. Lip. Res. (1981) 22: 800-810). Liver perfusion studies demonstrate that apo CIII inhibits the hepatic uptake of triglyceride-rich lipoproteins (TRL) and their remnants (Shelburne et al., J. Clin. Inves., (1980) 65: 652-658, Windler et al., J. Lip. Res. (1985) 26: 556-563). Also in vitro experiments show that apo CIII inhibit the activity of both lipoprotein lipase (LPL) and hepatic lipase (Brown and Bakinsky, Biochim. Biophs. Acta. (1972) 46: 375-382; Krauss et al., Circ. Res. (1973) 33: 403-411; Wang et al., J. Clin. Inves. (1985) 75: 384-390; McConathy et al., J. Lip. Res. (1972) 33: 995-1003; Kinnemen and Enholm, FEBS (1976) 65: 354-357). Moreover, ApoCIII is said to be involved in inhibition of LDL binding to LDL receptors (Fruchart et al. supra), via ApoB.

The role of apo CIII in plasma TRL metabolism has been more defined by the results of recent studies in transgenic animals (Aalto-Setälä et al., J. Clin. Invest. (1992) 90:5 1889-1900.). Plasma accumulation of TRL in mice overexpressing apo CIII has been shown to be associated with reduced plasma VLDL and chylomicron clearance (Harrold et al., J. Lip. Res. (1996) 37: 754-760) also the inhibitory effect of C apolipoproteins on the LDL receptor of apo B-containing lipoproteins was demonstrated (Clavey et al., Arth. Thromb. and Vase. Biol. (1995) 15: 963-971).

Previous vaccines in the field of immunotherapy of atherosclerosis have focused on the use of cholesterol as an immunogen to reduce serum cholesterol levels (Bailey, J. M. et al., 1994, Biochemical Society Transactions, 22, 433S; Alving, C. and Swartz, G. M., 1991, Crit. Rev. Immunol., 10, 441-453; Alving, C. and Wassef, N. M., 1999, Immunology Today, 20, 8, 362-366). Others have attempted to alter the activity of the Cholesterol Ester Transfer Protein (CETP) by vaccination (WO 99/15655). Alternatively, some authors have described vaccines using oxidised LDL as the immunogen, in order to inhibit plaque formation after balloon injury in hypercholesterolemic rabbits (Nilsson, J. et al., 1997, JACC, 30, 7, 1886-1891).

It has been found, surprisingly, that atherosclerosis may be prevented or ameliorated by active or passive immunotherapy, by reducing or blocking the function of ApoCIII. In particular, the active or passive immunotherapies of the present invention can be advantageously carried out using epitopes of ApoCIII. The use of peptides of ApoCIII comprising useful epitopes can focus the immune response to parts of the human ApoCIII molecule without triggering a general response to the whole molecule. Without wishing to be bound by theory, this can not only reduce non-preventative immune reactions against human ApoCIII, it can also be used as a means of distinguishing parts of ApoCIII that are surface exposed on LDL and not HDL, thus focusing the immune response against carriers of “bad cholesterol”, whilst not effecting the positive role of ApoCIII in HDL.

The active or passive immunotherapies of the present invention target an epitope found within the region between amino acid number 12 and 35, or an epitope found within the region between amino acids 45 and 76 of the human ApoCIII molecule as it exists in the circulation of a human, in addition it is preferred that the immunotherapy targets the epitope that is found within the region between amino acid 12 to 21 or 45 to 65 of human ApoCIII.

The sequence of the region between amino acid number 12 and 35 of the human ApoCIII is as follows:

MQGYMKHATKTAKDALSSVQESQV (SEQ ID NO. 2).

The sequence of the region between amino acid number 12 and 21 of the human ApoCIII is as follows:

MQGYMKHATK (SEQ ID NO. 3)

The sequence of the region between amino acid number 45 and 76 of the human ApoCIII is as follows:

DGFSSLKDYWSTVKDKFSEFWDLDPEVRPTSA (SEQ ID NO: 4)

The sequence of the region between amino acid number 45 and 65 of the human ApoCIII is as follows:

DGFSSLKDYWSTVKDKFSEFW (SEQ ID NO: 5)

The present invention also provides the following fragments of the above peptides within which contain an epitope of ApoCIII which may be targeted by the active or passive immunotherapies of the present invention:

| Peptide Sequence | SEQ ID NO: |
|---|---|
| MQGYMKHA | 6 |
| QGYMKHAT | 7 |
| GYMKHATK | 8 |
| YMKHATKT | 9 |
| MKHATKTA | 10 |
| KHATKTAK | 11 |
| HATKTAKD | 12 |
| ATKTAKDA | 13 |
| TKTAKDAL | 14 |
| KTAKDALS | 15 |
| TAKDALSS | 16 |
| AKDALSSV | 17 |
| KDALSSVQ | 18 |
| DALSSVQE | 19 |
| ALSSVQES | 20 |
| LSSVQESQ | 21 |
| SSVQESQV | 22 |
| DGFSSLKD | 23 |
| GFSSLKDY | 24 |
| FSSLKDYW | 25 |
| SSLKDYWS | 26 |
| SLKDYWST | 27 |
| LKDYWSTV | 28 |
| KDYWSTVK | 29 |
| DYWSTVKD | 30 |
| YWSTVKDK | 31 |
| WSTVKDKF | 32 |
| STVKDKFS | 33 |
| TVKDKFSE | 34 |
| VKDKFSEF | 35 |
| KDKFSEFW | 36 |
| DKFSEFWD | 37 |
| KFSEFWDL | 38 |
| FSEFWDLD | 39 |
| SEFWDLDP | 40 |
| EFWDLDPE | 41 |
| FWDLDPEV | 42 |
| WDLDPEVR | 43 |
| DLDPEVRP | 44 |
| LDPEVRPT | 45 |
| DPEVRPTS | 46 |
| PEVRPTSA | 47 |

The present invention provides vaccine immunogens effective in the prophylaxis or therapy of atherosclerosis which comprise immunogens that raise an immune response against the epitopes listed in SEQ ID NOS. 2-47, of ApoCIII, and also provides for methods of treatment of atherosclerosis by the administration of the immunogens of the present invention to individuals in need thereof. Most preferably the immunogens of the invention comprise the epitopes listed in SEQ ID NOS: 2, 3, 6-22. Preferably, the immunogens of the invention do not comprise the full length human ApoCIII sequence (SEQ ID NO: 1).

The present invention also provides monoclonal antibodies that are specific for the epitopes described in SEQ ID NOS. 2-47. Also provided are methods of treatment of individuals by passive administration of the monoclonal antibodies to the individual.

Active Immunotherapy

In the first aspect of the present invention, the immunogens of the present invention are capable of generating immune responses that recognise the epitopes SEQ ID NOS. 2-47 (preferably in the context of the mature human ApoCIII molecule). Accordingly, the immunogens may comprise or contain SEQ ID NO'S. 2-47, or they may comprise or contain synthetic peptides having the sequences listed in SEQ ID NOS. 2-47, or the immunogens may comprise or contain mimotopes thereof which retain the functional activity of being able to induce immune responses that recognise the epitopes listed in SEQ ID NOS. 2-47 preferably in the context of the mature human ApoCIII molecule). Most preferably the immunogens of the invention comprise the epitopes listed in SEQ ID NOS: 2, 3, 6-22. Preferably, the immunogens of the invention do not comprise the full length human ApoCIII sequence (SEQ ID NO: 1).

Most preferably the antibodies induced by the immunogens of the present invention are functional in the treatment of atherosclerosis, and in a preferred form of the present invention they abrogate the inhibition exerted by ApoCIII on the binding of ApoB to its receptor, and/or the activity of lipoprotein lipase. Such activities may readily be assayed by the man skilled in the art for example by methods described in Fruchard et al, supra; and McConathy et al., supra.

The immunogen may comprise or contain the full length peptides of SEQ ID NOS. 2-47, or alternatively the immunogen may comprise or contain fragments of the identified peptides, lacking 1, 2, 3, 5 or 10 amino acids from either or both of the N- or C-termini of the peptides. Alternatively, the immunogen may comprise or contain a peptide which is longer than SEQ ID NOS. 2-47, that contain SEQ ID NOS. 2-47 within the longer sequence. Preferably, peptides with 1, 2, 3, 5, 10, or 20 amino acids may be added to either or both of the N- or C-termini of the peptides from the native context of the peptides within human mature ApoCIII. Most preferably, in this case, the longer immunogens are less than 80 amino acids in length, more preferably less than 50 amino acids, more preferably less than 40 amino acids and most preferably less than 25 amino acids long. Preferably, the immunogens of the invention do not comprise the full length human ApoCIII sequence (SEQ ID NO: 1). The immunogen may be longer than those described above if it further comprises a carrier molecule fused to the peptides of the invention as described below.

In yet another alternative, the immunogen may be a true mimotope of the linear sequences described in SEQ ID NOS. 2-47, in that the sequence of the peptide mimotope is not-necessarily related to the sequences of SEQ ID NOS. 2-47, but may represent a three dimensional conformational epitope which binds to the region corresponding to the folded tertiary structure of ApoCIII which is made up of the amino acids of SEQ ID NOS. 2-47.

The immunogens of the present invention may, therefore, comprise or contain the isolated peptides encompassing the apolipoprotein epitopes themselves, and any mimotope thereof. The meaning of mimotope is defined as an entity which is sufficiently similar to the apolipoprotein epitope so as to be capable of being recognised by antibodies which recognise the apolipoprotein; (Gheysen, H. M., et al., 1986, Synthetic peptides as antigens. Wiley, Chichester, Ciba foundation symposium 119, p130-149; Gheysen, H. M., 1986, Molecular Immunology, 23, 7, 709-715); or are capable of raising antibodies, when coupled to a suitable carrier, which antibodies cross-react with the native apolipoprotein.

Peptide mimotopes of the above-identified ApoCIII peptides/epitopes may be designed for a particular purpose by addition, deletion or substitution of elected (1, 2, 3, 4, 5 or more) amino acids. Thus, the peptides of the present invention may be modified for the purposes of ease of conjugation to a protein carrier. For example, it may be desirable for some chemical conjugation methods to include a terminal (N- and/or C-) cysteine to the apolipoprotein epitope. In addition it may be desirable for peptides conjugated to a protein carrier to include a hydrophobic terminus distal from the conjugated terminus of the peptide, such that the free unconjugated end of the peptide remains associated with the surface of the carrier protein. This reduces the conformational degrees of freedom of the peptide, and thus increases the probability that the peptide is presented in a conformation which most closely resembles that of the apolipoprotein peptide as found in the context of the whole apolipoprotein. For example, the peptides may be altered to have an N-terminal cysteine and a C-terminal hydrophobic amidated tail. Conformational restriction may also take place if N- and C-termini of the peptides are Cysteine residues which may be induced to form a cyclised peptide through a disulphide bond (optionally having additional terminal amino acids for conjugation to a carrier molecule). D and K residues may also be included at N- and C-termini of the peptides of the invention, respectively (or vice versa), in order to form cyclised peptides via a β-lactam bond which can be straightforwardly made between D and K residues (optionally such peptides may have an additional terminal amino acid [such as a Cysteine] for conjugation to a carrier molecule. Alternatively, the addition or substitution of a D-stereoisomer form of one or more of the amino acids may be performed to create a beneficial derivative, for example to enhance stability of the peptide. Those skilled in the art will realise that such modified peptides, or mimotopes, could be a wholly or partly non-peptide mimotope wherein the constituent residues are not necessarily confined to the 20 naturally occurring amino acids. In addition, these may be cyclised by techniques known in the art to constrain the peptide into a conformation that closely resembles its shape when the peptide sequence is in the context of the whole apolipoprotein (for example by the addition of a cysteine at the terminal regions of the peptide to form a disulphide bridge).

The peptide mimotopes may also be retro sequences of the natural apolipoprotein peptide sequences, in that the sequence orientation is reversed; or alternatively the sequences may be entirely or at least in part comprised of D-stereo isomer amino acids (inverso sequences). Also, the peptide sequences may be retro-inverso in character, in that the sequence orientation is reversed and the amino acids are of the D-stereoisomer form. Such retro or retro-inverso peptides have the advantage of being non-self, and as such may overcome problems of self-tolerance in the immune system.

Alternatively, peptide mimotopes may be identified using antibodies which are capable themselves of binding to the apolipoprotein, using techniques such as phage display technology (EP 0 552 267 B 1). This technique, generates a large number of peptide sequences which mimic the structure of the native peptides and are, therefore, capable of binding to anti-native peptide antibodies, but may not necessarily themselves share significant sequence homology to the native apolipoprotein.

Particularly preferred peptides of the present invention are any peptide that is capable of binding to the antibodies deposited under the provisions of the Budapest Treaty for deposits of biological material, on the Aug. 1^{st} 2001, at ECACC (European Collection of Cell Cultures, Vaccine Research and Production Laboratory, Public Health Laboratory Service, Centre for Applied Microbiology Research, Porton Down, Salisbury, Wiltshire, SP4 OJG, UK), under the accession numbers 01080123 (ApoCIII/4IIa), 01080122 (ApoCIII/5IIa), 01080121 (ApoCIII/10IIa), 01080120 (ApoCIII/12IIa), 01080124 (ApoCIII/13IIa). Alternatively, peptides of the present invention (which may be used to form immunogens and vaccines of the present invention) include any peptide that is capable of competing with ApoCIII for binding to the above deposited monoclonal antibodies.

In the vaccines of the present invention the epitope or mimotope is preferably linked to a carrier molecule to form an immunogen which enhances the immunogenicity of the epitope. Accordingly, the peptides or mimotopes may be linked via chemical covalent conjugation or by expression of genetically engineered fusion partners, optionally via a linker sequence. The peptides may have two or more Glycine residues as a linker sequence, and often have a terminal exposed cysteine residue for linkage purposes.

The covalent coupling of the epitope of ApoCIII, to the carrier protein can be carried out in a manner well known in the art. Thus, for example, for direct covalent coupling it is possible to utilise a carbodiimide, glutaraldehyde or (N-[γ-maleimidobutyryloxy]) succinimide ester, utilising common commercially available heterobifunctional linkers such as CDAP and SPDP (using manufacturers instructions).

The types of carriers used in the immunogens of the present invention will be readily known to the man skilled in the art. The function of the carrier is to provide cytokine help (or T-cell help) in order to enhance the immune response against the apolipoprotein or apolipoprotein peptide. A non-exhaustive list of carriers which may be used in the present invention include: Keyhole limpet Haemocyanin (KLH), serum albumins such as bovine serum albumin (BSA), inactivated bacterial toxins such as tetanus or diptheria toxins (TT and DT, or the DT derivative CRM197), or recombinant fragments thereof (for example, Domain 1 of Fragment C of TT, or the translocation domain of DT), or the purified protein derivative of tuberculin (PPD). Alternatively the epitopes or may be linked to the carrier in a non-covalent fashion such as association via a liposome carrier or by co-adsorption onto an aluminium salt, which may additionally comprise immunogens capable of providing T-cell help or additional adjuvant immunostimulators. Preferably the ratio of the number of apolipoprotein, or fragment or peptide thereof, to carrier protein is in the order of 1:1 to 20:1, and preferably each carrier should carry between 3-15 apolipoproteins, or peptide or fragment thereof.

In an embodiment of the invention the carrier is Protein D from Haemophilus influenzae (EP 0 594 610 B1). Protein D is an IgD-binding protein from Haemophilus influenzae and has been patented by Forsgren (WO 91/18926, granted EP 0 594 610 B1). In some circumstances, for example in recombinant immunogen expression systems it may be desirable to use fragments of protein D, for example Protein D ⅓^{rd} (comprising the N-terminal 100-110 amino acids of protein D (WO 99/10375; WO 00/50077)).

Another preferred method of presenting the peptides of the present invention, is in the context of a recombinant fusion molecule. For example, EP 0 421 635 B describes the use of chimeric hepadnavirus core antigen particles to present foreign peptide sequences in a virus-like particle. As such, immunogens of the present invention may comprise the epitopes described in SEQ ID NOS. 2-47, or fragments or mimotopes thereof, presented in chimeric particles consisting of hepatitis B core (HepB core) antigen. Additionally, the recombinant fusion proteins may comprise the mimotopes of the present invention and a carrier protein, such as NS1 of the influenza virus. For any recombinantly expressed protein which forms part of the present invention, the nucleic acid which encodes said immunogen also forms an aspect of the present invention. Preferably, a recombinant fusion of 2, 3, 4 or more peptides, with at least one carrier fused in between the peptides, is used in the present invention.

Accordingly, preferred immunogens of the present invention comprise the epitope SEQ ID NO: 2-47, presented in a recombinant expression system (such as HepB core) or conjugated to a carrier protein, such that the recombinant expression system or the carrier protein provide T-cell help for generation of an immune response to SEQ ID NO: 2-47, respectively, (preferably against SEQ ID NO: 2 or 3 for immunogens based on SEQ ID NO: 2, 3, 6-22, and against SEQ ID NO: 4 or 5 for immunogens based on SEQ ID NO: 4, 5, 23-47).

In an alternative embodiment of the present invention the immunogenicity of the peptides is enhanced by the addition of T-helper (Th) epitopes. The immunogens of the present invention may, therefore, comprise the peptides as described previously and promiscuous Th epitopes either as chemical or recombinant conjugates or as purely synthetic peptide constructs. The apolipoprotein peptides are preferably joined to the Th epitopes via a spacer (e.g., Gly-Gly) at either the N- or C-terminus of the apolipoprotein peptide. The immunogens may comprise 1 or more promiscuous Th epitopes, and more preferably between 2 to 5 Th epitopes.

A Th epitope is a sequence of amino acids that comprise a Th epitope. A Th epitope can consist of a continuous or discontinuous epitope. Hence not every amino acid of Th is necessarily part of the epitope. Th-epitopes that are promiscuous are highly and broadly reactive in animal and human populations with widely divergent MHC types (Partidos et al. (1991) “Immune Responses in Mice Following Immunization with Chimeric Synthetic Peptides Representing B and T Cell Epitopes of Measles Virus Proteins” J. of Gen. Virol. 72:1293-1299; U.S. Pat. No. 5,759,551). The Th domains that may be used in accordance with the present invention have from about 10 to about 50 amino acids, and preferably from about 10 to about 30 amino acids. When multiple Th epitopes are present, each Th epitope is independently the same or different.

Th epitopes include as examples, pathogen derived epitopes such as Hepatitis surface or core (peptide 50-69, Ferrari et al., J. Clin. Invest, 1991, 88, 214-222) antigen Th epitopes, Pertussis toxin Th epitopes, tetanus toxin Th epitopes (such as P2 (EP 0 378 881 B1) and P30 (WO 96/34888, WO 95/31480, WO 95/26365), measles virus F protein Th epitopes, Chlamidia trachomatis major outer membrane protein Th epitopes (such as P11, Stagg et al., Immunology, 1993, 79, 1-9), Yersinia invasin and diptheria toxin Th epitopes. Other Th epitopes are described in U.S. Pat. No. 5,759,551 and Cease et al., 1987, Proc. Natl. Acad. Sci. 84, 4249-4253; and Partidos et al., J. Gen. Virol, 1991, 72, 1293-1299; WO 95/26365 and EP 0 752 886 B.

In one embodiment of the invention, the carrier is a tetanus toxin (TT) peptide T-helper epitope. Examples of TT peptides which may be used as carriers in the present invention include the P2 peptide of TT and the P30 epitope of TT. The TT peptide carriers may be linked to whole Apo CIII (SEQ ID NO 1) or fragments thereof, as described herein. Preferably, the TT peptide carriers are linked to immunogens which comprise or contain the sequence listed in any of SEQ ID NO: 2-47 or truncations or extensions thereof as described herein. The link between the TT peptide carrier and the immunogen may be through the C-terminus or the N-terminus. Alternatively, the TT peptide carrier may be linked, preferably fused, between two or more peptides of Apo CIII or fragments thereof, i.e. one or more ApoCIII peptides each side of a TT peptide carrier. Examples of preferred embodiments are listed below. It will be appreciated that the examples are not an exhaustive list; any combination of whole or fragmented Apo CIII and TT T-helper epitope peptides which enhance immunogenicity of the Apo CIII epitope are included in the present invention.

(i). ApoCIII-P2

=whole ApoCIII (SEQ ID NO 1) linked to P2 (peptide of TT) at C-term

(ii). P2-ApoCIII

=whole ApoCIII (SEQ ID NO 1) linked to P2 (peptide of TT) at N-term

(iii). (12-35)-P2-(12-35)

=P2 between two peptides (SEQ ID NO 2) of ApoCIII

(iv). (12-35)-P30-(12-35)

=P30 (peptide of TT) between two peptides (SEQ ID NO 2) of ApoCIII

(v). (45-65)-P2-(45-65)

=P2 between two peptides (SEQ ID NO 5) of ApoCIII

(vi). (45-76)-P2-(45-76)

=P2 between two peptides (SEQ ID NO 4) of ApoCIII

(vii). (45-65)-P30-(45-65)

=P30 between two peptides (SEQ ID NO 5) of ApoCIII

(viii). (45-76)-P30-(45-76)

=P30 between two peptides (SEQ ID NO 4) of ApoCIII

The advantage of linking more than one immunogen to a carrier is this provides a greater immune response to the epitopes of the immunogen. The advantage of using P2 or P30 as peptide carriers, is these specific carriers greatly enhance the immune response to the epitopes of the present invention.

The immunogens of the present invention are provided for use in medicine, for use in the treatment or prevention of atherosclerosis, and for formulation into immunogenic compositions or vaccines of the present invention.

Another preferred epitope that forms part of the present invention is the peptide found between amino acids 21 and 35 of human ApoCIII.

The immunogenic compositions and vaccines comprise one or more immunogens of the present invention as previously described, and may advantageously also include an adjuvant. Suitable adjuvants for vaccines of the present invention comprise those adjuvants that are capable of enhancing the antibody responses against the apolipoprotein immunogen. Adjuvants are well known in the art (Vaccine Design—The Subunit and Adjuvant Approach, 1995, Pharmaceutical Biotechnology, Volume 6, Eds. Powell, M. F., and Newman, M. J., Plenum Press, New York and London, ISBN 0-306-44867-X). Preferred adjuvants for use with immunogens of the present invention include: aluminium or calcium salts (hydroxide or phosphate), oil in water emulsions (WO 95/17210, EP 0 399 843), or particulate carriers such as liposomes (WO 96/33739). Immunologically active saponin fractions (e.g. Quil A) having adjuvant activity derived from the bark of the South American tree Quillaja Saponaria Molina are particularly preferred. Derivatives of Quil A, for example QS21 (an HPLC purified fraction derivative of Quil A), and the method of its production is disclosed in U.S. Pat. No. 5,057,540. Amongst QS21 (known as QA21) other fractions such as QA17 are also disclosed. 3 De-O-acylated monophosphoryl lipid A (3D-MPL) is a well known adjuvant manufactured by Ribi Immunochem, Montana. It can be prepared by the methods taught in GB 2122204B. A preferred form of 3D-MPL is in the form of an emulsion wherein the 3D-MPL has a small particle size of less than 0.2 μm in diameter (EP 0 689 454 B1). Other non-toxic derivatives of Lipid A may also be used.

Adjuvants also include, but are not limited to, muramyl dipeptide and saponins such as Quil A, bacterial lipopolysaccharides such as 3D-MPL (3-O-deacylated monophosphoryl lipid A), or TDM. As a further exemplary alternative, the protein can be encapsulated within microparticles such as liposomes, or in non-particulate suspensions of polyoxyethylene ether (WO 99/52549). Particularly preferred adjuvants are combinations of 3D-MPL and QS21 (EP 0 671 948 B1), oil in water emulsions comprising 3D-MPL and QS21 (WO 95/17210, PCT/EP98/05714), 3D-MPL formulated with other carriers (EP 0 689 454 B1), or QS21 formulated in cholesterol containing liposomes (WO 96/33739), or immunostimulatory oligonucleotides (WO 96/02555).

The vaccines of the present invention will be generally administered for both priming and boosting doses. It is expected that the boosting doses will be adequately spaced, or preferably given yearly or at such times where the levels of circulating antibody fall below a desired level. Boosting doses may consist of the peptide in the absence of the original carrier molecule (or Th epitope). Such booster constructs may comprise an alternative carrier (or Th epitope) or may be in the absence of any carrier (or Th epitope).

In a further aspect of the present invention there is provided a vaccine or immunogenic composition as herein described for use in medicine.

The immunogenic composition or vaccine preparations of the present invention may be used to protect or treat a mammal susceptible to, or suffering from atherosclerosis, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts.

The amount of protein in each vaccine or immunogenic composition dose is selected as an amount which induces an immunoprotective response without significant, adverse side effects in typical vaccinees. Such amount will vary depending upon which specific immunogen is employed and how it is presented. Generally, it is expected that each dose will comprise 1-1000 μg of protein, preferably 1-500 μg, preferably 1-100 μg, of which 1 to 50 μg is the most preferable range. An optimal amount for a particular vaccine can be ascertained by standard studies involving observation of appropriate immune responses in subjects. Following an initial vaccination, subjects may receive one or several booster immunisations adequately spaced.

Vaccine preparation is generally described in New Trends and Developments in Vaccines, edited by Voller et al., University Park Press, Baltimore, Md., U.S.A. 1978. Conjugation of proteins to macromolecules is disclosed by Likhite, U.S. Pat. No. 4,372,945 and by Armor et al., U.S. Pat. No. 4,474,757.

Passive Immunotherapy

In a second aspect of the present invention are monoclonal Ab's capable of binding to epitopes of SEQ ID NOS. 2 to 47 (preferably SEQ ID NO: 2 or 3) in the context of the human ApoCIII molecule, and their use in immunotherapy.

Monoclonal antibodies that recognise the region 12-35 of human ApoCIII are ApoCIII/4IIa, ApoCIII/5IIa, ApoCIII/10IIa, ApoCIII/12IIa and ApoCIII/13IIa. The hybridomas for these monoclonal antibodies are deposited under the provisions of the Budapest Treaty for deposits of biological material, on the Aug. 1^{st} 2001, at ECACC (European Collection of Cell Cultures, Vaccine Research and Production Laboratory, Public Health Laboratory Service, Centre for Applied Microbiology Research, Porton Down, Salisbury, Wiltshire, SP4 OJG, UK), under the accession numbers 01080123 (ApoCIII/4IIa), 01080122 (ApoCIII/5IIa), 01080121 (ApoCIII/10IIa), 01080120 (ApoCIII/12IIa), 01080124 (ApoCIII/13IIa).

The protein sequences of these monoclonal antibodies, and therefore the sequences of the hypervariable regions and the CDR's is fully encompassed within the present invention, as it can be readily be obtained by sequencing of the deposited antibody and/or sequencing of the hybridoma genome using techniques well know to the man skilled in the art.

Also encompassed within the scope of the present invention are “similar” antibodies to the above identified deposited monoclonal antibodies. For example, the present invention also provides other antibodies that recognise the same epitope as the deposited antibodies. The same recognition may be assayed by competition ELISA where the “similar” monoclonal” competes with the deposited antibody for binding to ApoCIII. Alternatively the similar antibody may have a similar or identical amino acid sequence in its hypervariable regions, and/or the same or similar complementarity determining regions (CDR), so that the antibody is capable of competing with the deposited antibody for binding to ApoCIII. Additionally, “humanised” or “fully human” versions of these deposited murine antibodies, which contain the same or similar CDRs as the deposited antibodies, are also encompassed within the scope of the present invention. A fully human version can be obtained, for instance, by immunising a transgenic mouse having a set of human antibody-encoding genes with the immunogenic compositions of the invention.

The term “antibody” herein is used to refer to a molecule having a useful antigen binding specificity. Those skilled in the art will readily appreciate that this term may also cover polypeptides which are fragments of or derivatives of antibodies yet which can show the same or a closely similar functionality. Such antibody fragments or derivatives are intended to be encompassed by the term antibody as used herein.

The term “monoclonal antibody” is used herein to encompass any isolated Ab's such as conventional monoclonal antibody hybridomas, but also to encompass isolated monospecific antibodies produced by any cell, such as for example a sample of identical human immunoglobulins expressed in a mammalian cell line.

The monoclonal antibodies of the present invention are capable of being used in passive prophylaxis or therapy, by administration of the antibodies into a patient, for the amelioration of atherogenic disease.

The monoclonal antibodies of the present invention may be generated using the immunogens of the present invention (using known techniques e.g. Köhler and Milstein, Nature, 1975, 256, p495).

Also, there is provided by the present invention, an isolated antibody generated against the immunogens of the present invention.

Hybridomas secreting the monoclonal antibody ligands of the present invention are also provided.

Pharmaceutical compositions comprising the ligands, described above, also form an aspect of the present invention. Also provided are the use of the ligands in medicine, and in the manufacture of medicaments for the treatment of atherosclerosis.

In the passive treatments of atherosclerosis as provided herein, the administration of the ligands or antibodies of the present invention will be administered (preferably intra-venously) to the patients in need thereof. The frequency of administration may be determined clinically by following the decline of antibody titres in the serum of patients over time, but in any event may be at a frequency of 1 to 52 times per year, and most preferably between 1 and 12 times per year. Quantities of antibody or ligand may vary according to the severity of the disease, or half-life of the antibody in the serum, but preferably will be in the range of 1 to 10 mg/kg of patient, and preferably within the range of 1 to 5 mg/kg of patient, and most preferably 1 to 2 mg/kg of patient.

The immunogens, immunogenic compositions, vaccines or monoclonal antibodies of the present invention may be administered to a patient who is suffering from, or is at risk to, atherosclerotic disease, and are effective in re-establishing the correct equilibrium of the “bad” lipoproteins (apo B containing lipoproteins) to the “good ” lipoproteins (apo A-I containing lipoproteins) balance, and minimise the circulation time of apo B containing lipoproteins. Not wishing to be bound by theory, the inventors believe that these functions minimise the possibility of deposit and oxidation of apo B containing lipoproteins within the blood vessel walls, and hence, reduce the risk of atherosclerotic plaque formation or growth.

The present invention, therefore, provides the use of the ApoCIII epitopes, ligands (monoclonal antibodies) and immunogens of the present invention (as defined above), in the manufacture of pharmaceutical compositions for the prophylaxis or therapy of atherosclerosis. Accordingly, the ApoCIII immunogens of the present invention are provided for use in medicine, and in the medical treatment or prophylaxis of atherosclerosis.

There is also provided a method of treatment or prophylaxis of atherosclerosis comprising the administration to a patient suffering from or susceptible to atherosclerosis, of an immunogenic composition or vaccine or ligand of the present invention.

A method of prophylaxis or treatment of atherosclerosis is provided which comprises a reduction of total circulating triglyceride levels in a patient, by the administration of a vaccine of the present invention to the patient. In particular there is provided a method of reducing the amount of circulating VLDL and LDL in a patient, by the administration of the vaccine or ligands of the present invention to the patient.

Also provided is a method of prophylaxis or treatment of atherosclerosis by the administration to a patient of a vaccine which is capable of reducing the average circulation time of ApoB containing lipoproteins. In this regard the average circulation time of ApoB containing lipoproteins, may be investigated in an in vivo animal model by the measuring the clearance rate of labelled ApoB containing lipoproteins from the plasma of the mammal (half-life of labelled ApoB containing lipoproteins).

A preferred immunogen for these method of treatment aspects of the present invention comprises or contains the ApoCIII epitopes SEQ ID NO: 2-47 (preferably SEQ ID NO: 2 or 3). Surprisingly, the targetting of ApoCIII by the vaccine or the monoclonal Ab downregulates the negative effects of the “bad” cholesterol (LDL), whilst not having a negative effect on the “good” cholesterol (HDL).

Preferred methods of treating individuals suffering from Atherosclerosis having elevated levels of circulating ApoCIII in their plasma comprise reducing the levels of circulating ApoCIII, by the administration of a vaccine comprising or containing the ApoCIII epitope SEQ ID NO: 2-47 (preferably SEQ ID NO: 2 or 3), or mimotope thereof, as an immunogen to said individual. Alternatively, in a related aspect of the present invention there is provided a method of treatment or prophylaxis of atherosclerosis by reducing the levels of circulating ApoCIII in the plasma of a patient, by administration of a monoclonal Ab that is capable of blocking the activity of ApoCIII, by binding to the epitope SEQ ID NO: 2-47 (preferably SEQ ID NO. 2 or 3) and thereby abrogating the ApoCIII-mediated inhibition of lipoprotein lipase and/or the binding of ApoB to its receptor, to said patient.

Also provided by the present invention is a method of treatment or prophylaxis of atherosclerosis by reducing the number of ApoCIII molecules which are associated with an ApoB molecule in situ in the context of a lipoprotein by administration of a monoclonal Ab, or vaccine of the present invention. In a normal individual there is approximately one ApoB present in an LDL particle, the ApoB being associated with between 1-5 ApoCIII molecules. In diseased individuals the number of ApoCIII molecules may increase to up to 25. Accordingly, there is provided by the present invention a method of treatment or prophylaxis of atherosclerosis by reducing the ratio of ApoCIII molecules per ApoB molecules in the LDL in an individual with atherosclerosis from a high disease state level (approximately 20 to 25:1) to a reduced therapeutic level preferably below 15:1, more preferably below 10:1 and more preferably below 5:1, preferably below 3:1, and most preferably approximately 1:1 ApoC:ApoB. Levels of ApoCIII contained within ApoB-containing lipoproteins may be measured by nephelometry or electro-immunodiffusion (normal range is 2 to 3 mg/dL).

Also provided by the present invention is a combination therapy for treatment or prophylaxis of atherosclerosis comprising the active or passive immunotherapy of the present invention in combination with any other therapy or combination of therapies for treatment or prophylaxis of atherosclerosis, such as immunotherapy directed towards modified ApoCIII, oxidised ApoA, oxidised ApoB (as described in WO02/080954), oxidised LDL (WO02/50550) or Cholesterol Ester Transfer Protein (CETP; WO99/15655), or other known therapies.

The present invention is illustrated, but not limited, by the following examples:

### EXAMPLES

### Example 1

Peptide Synthesis

The ApoCIII peptides (1-79, 12-21, 12-35, 45-65, 19-28, 26-35, 1-17, 17-24 and 45-76 were synthesised by the solid phase method (Merrifield, 1986) on an automated synthesiser Model ABI 433A (Applied Biosystems Inc.) using Boc/Bzl strategy on a Boc-Ala-PAM resin for total apo CIII and MBHA resin for the others fragments. Each amino acid was coupled twice by dicyclohexylcarbodiimide/hydroxybenzotriazole without capping. Side chain protecting groups were as follows: Arg(Ts), Asp(Ochex), Glu(Ochex), Lys(2-Cl-Z), His(Dnp), Ser(Bzl), Thr(Bzl), Met(O) and Tyr(Br-Z). According to the sequence, the group Dnp on His was removed from the peptide, prior to the cleavage from its support by treatment with 10% β-mercaptoethanol, 5% diisopropylethylamine in DCM for 2 h and in NMP for 2 h. The peptidyl resin was then treated with 50% TFA in DCM for 20 min to remove the amino-terminal Boc. The peptide was cleaved from the resin and simultaneously deprotected according to a low and high HF procedure: the resin (1 g) was treated with anhydrous HF (2.5 mL) in the presence of p-cresol (0.75 g), p-thiocresol (0.25 g) and dimethylsulfide (6.5 mL) at 0° C. After 3 h hydrogen fluoride and dimethylsulfide were removed by vacuum evaporation and the residual scavengers and by products were extracted with diethyl ether. The reaction vessel was recharged with p-cresol (0.75 g), p-thiocresol (0.25 g) and 10 ml of anhydrous HF and the mixture was allowed to react at 0° C. for 1.5 h. Hydrogen fluoride was removed by evaporation and the residue was triturated with diethyl ether. The residue was filtered off, washed with diethyl ether and extracted with 200 ml of 10% aqueous acetic acid and lyophilised. The crude product was analysed by reversed-phase HPLC on a Vydac C18 column (4.6×250 mm, 5μ, 100 A) using 60 min linear gradient from 0 to 100% Buffer B (Buffer A: 0.05% TFA in H₂O and Buffer B: 0.05% TFA, 60% CH₃CN in H₂O) at flow rate of 0.7 ml/min and detection was performed at 215 nm. Synthetic peptide were purified by RP-HPLC and were characterised and analysed by HPLC, the molecular mass determined by spectrometry.

### Example 2

Monoclonal Antibody Production

Peptides were synthesised as described in Example 1 with the addition of a small linker sequence (CGG) onto the carboxyl end of the peptide. The conjugate was produced using maleimide chemistry, by reacting this modified sequence with a commercial pre-activated BSA. BSA was purchased from Pierce, which was pre-activated with a succinimidyl 4-(N-maleimidomethyl)-cyclohexane-1-carboxylate (SMCC) linker. SMCC may also be bought from any major manufacturer and used following the manufacturers instructions. The coupling of the BSA to the peptide via the SMCC was carried out over 2 hr at room temperature with an excess of peptide, before quenching with the reaction with excess cystein, followed by dialysis against phosphate buffer.

A group of BalbC mice were immunised with 25 μg of conjugate BSA-peptide 12-35 formulated in an oil in water emulsion described in WO 95/17210. Intra muscular injections done at day 0, 14, 28.

Sera from the mice were evaluated by ELISA for strongest anti-peptide 12-35 and anti-complete ApoCIII responses.

Another functional assay was performed by ELISA to identify the mouse with the highest anti-ApoCIII titres when ApoCIII was in its native form and loaded into the lipoproteins. Briefly, plates were coated with affinity purified polyclonal antibodies to human ApoCIII. Plasma sample, HDL and VLDL particles were incubated, and after washing, revealed by sera of the immunized mice.

After a two-month resting period, the “best” mouse was boosted with antigen in saline and sacrificed three days later. Spleen cells were fused with the Sp2/0 B cell line according to standard protocols. First screening of hybridoma supernatants was performed by ELISA against the peptide 12-35 ApoCIII. Positive wells were subcloned and tested, this time also for reactivity against complete ApoCIII.

Five monoclonal hybridomas were obtained (No. 4 [or 4IIa], 5 [or 5IIa], 10 [or 10IIa], 12 [or 12IIa] and 13 [or 13IIa]) and deposited under the provisions of the Budapest Treaty for deposits of biological material, on the Aug. 1, 2001, at ECACC (European Collection of Cell Cultures, Vaccine Research and Production Laboratory, Public Health Laboratory Service, Centre for Applied Microbiology Research, Porton Down, Salisbury, Wiltshire, SP4 OJG, UK), under the accession numbers 01080123 (ApoCIII/4IIa), 01080122 (ApoCIII/5IIa), 01080121 (ApoCIII/10IIa), 01080120 (ApoCIII/12IIa), 01080124 (ApoCIII/13IIa).

### Example 3,

Characterisation of the Antibodies

Peptide specificity ELISA.: Microtiter plates (flat-bottom 96-well EIA; Costar, Dutscher) were washed with 0.1 mol/L phosphate-buffered saline (PBS, pH 7.2) before being coated with 100 μl/well of free peptide (5 μg/ml) (ApoCIII peptides produced in Example 1: 12-21, 12-35, 45-65, 45-76, 19-28, 26-35, 1-17, 17-24 and ApoCIII (1-79)) and incubated overnight at room temperature. The plates were washed four times with buffer and to minimise the non-specific binding to the microtiter wells, the plates were saturated with 250 μL/well of bovine serum albumin at 3% in 0.1 M PBS buffer and incubated for 1 h at 37° C. The plates were washed four times again and incubated for 2 h at 37° C. with 100 μL of the anti-12-35 monoclonal antibodies (produced in example 2), diluted in 1% of bovine serum albumin in 0.1 M PBS buffer, then washed three four times with PBS. To assess the immunological reaction, 100 μL of 10 000-fold diluted, anti-mouse IgG labelled with peroxidase, in 0.1 % of BSA in PBS buffer were added to each well. After an incubation for 2 h at 37° C., the plates were washed four times with PBS and 100 μL of substrate solution was added. The substrate solution was prepared as follows: 30 mg of o-Phenylenediamine dihydrochloride were dissolved in 20 ml of 0.1 mmol/L phosphate-citrate buffer, pH 5.5 containing 20 μL of 30% hydrogen peroxide. After 30 min at room temperature in the dark, the reaction was stopped by adding to each well 100 μl of 1 mmol/L HCl. The absorbance was measured at 492 nm.

Functional Assays

The objective was to check if the epitopes recognised by the monoclonal antibodies are accessible when ApoCIII is in the context of human plasma-purified lipoproteins (HDL, VLDL).

Sandwich ELISA: Microtiter plates (flat-bottom 96-well EIA; Costar, Dutscher) were washed with 0.1 mol/L phosphate-buffered saline (PBS, pH 7.2) before being coated

| | | | | | | | | | |
|---|---|---|---|---|---|---|---|---|---|
| 12-35TT | 8 | 25 | x | x | x | x | x | x | x |
| TT | 8 | 20 | x | x | x | x | x | x | x |

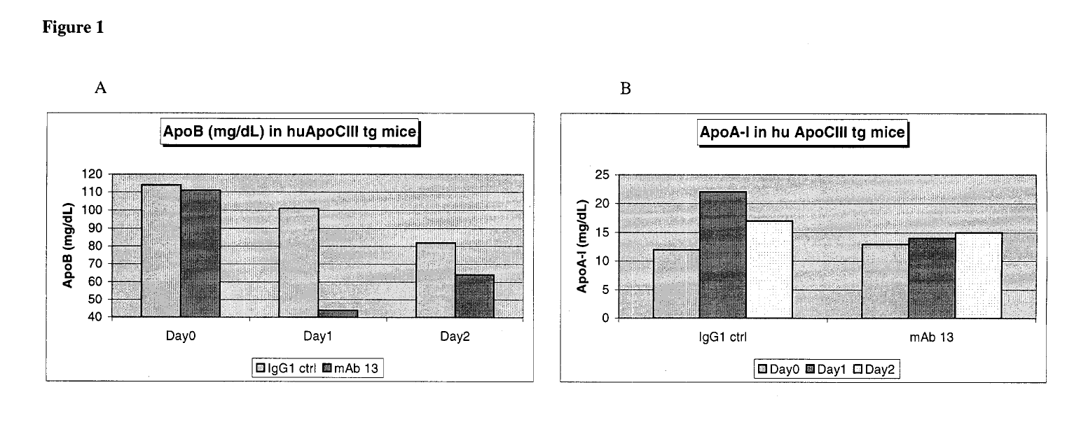

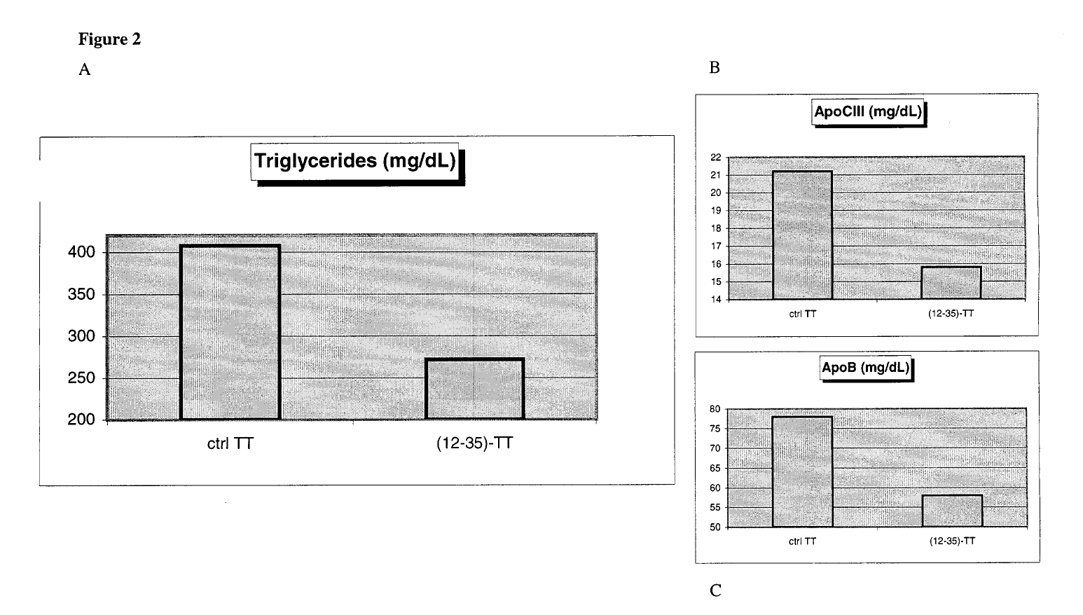

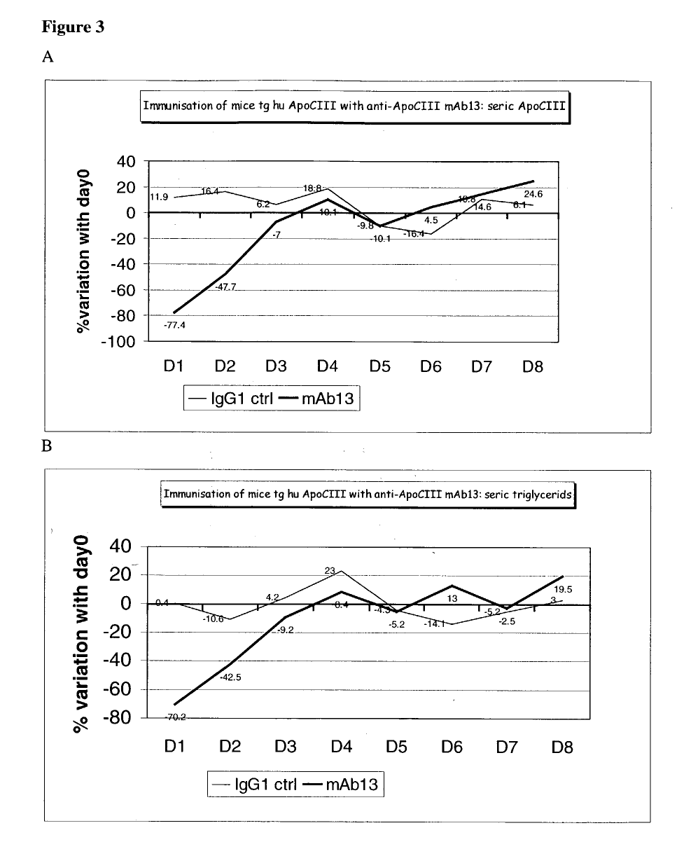

FIG. 2 shows the level (mg/dL) of triglycerides, ApoCIII and ApoB in the sera of the mice day 105 post the administration of the first dose of vaccine. As can be seen there is a significant concomitant decrease in the levels of these 3 molecules associated with VLDL and LDL. This is particularly significant when it is considered that the transgenic mice has 10 times the concentration of human ApoCIII of humans. The data also reflects the fact that a booster response (indicative of immune memory) occurred after the booster dose at Day 90.

Lipoprotein profiles carried out on the sera post boost indicated that the decrease in triglycerides was as a result of less being present in VLDL fractions. When cholesterol measurements were taken, however, it was observed that decreased cholesterol in sera was a result of less being present in VLDL fractions, but there was no change in the level of cholesterol in HDL fractions as compared to control sera. This is further evidence that the peptide 12-35 elicits an immune response that is specific for “bad cholesterol” containing lipoproteins.

1. A peptide comprising the epitope described in SEQ ID NO: 2, or mimotope or fragment thereof.

2. A vaccine immunogen comprising a peptide as claimed in claim 1, conjugated to a carrier molecule.

3. A vaccine immunogen according to claim 2 in which the link between the carrier and the peptide is through the C-terminus of the peptide.

4. A vaccine immunogen according to claim 2 in which the link between the carrier and the peptide is through the N-terminus of the peptide.

5. A vaccine immunogen according to claim 2 in which the carrier is linked to two or more peptides.

6. A vaccine immunogen according to claim 2 in which the carrier is recombinantly fused between two peptides.

7. A vaccine immunogen according to claim 2, wherein the carrier comprises a T-helper epitope.

8. A vaccine immunogen according to claim 2, wherein the carrier molecule is tetanus toxoid, or a peptide of tetanus toxin.

9. A vaccine immunogen according to claim 2, in which the carrier molecule is P2.

10. A vaccine immunogen according to claim 2, in which the carrier molecule is P30.

11. A vaccine immunogen according to claim 2 comprising n+1 peptides and n carriers, in which the peptides and carriers are alternately fused to each other, and in which n=1, 2, 3, 4, 5, 6 or 7.

12. A vaccine immunogen according to claim 11 in which all the peptides have the same sequence.

13. A vaccine immunogen according to claim 2 further comprising P2 fused between two peptides of SEQ ID NO 2 of ApoCIII

14. A vaccine immunogen according to claim 2 further comprising P30 (peptide of TT) between two peptides of SEQ ID NO 2 of ApoCIII

15. A vaccine immunogen according to claim 2 further comprising P2 between two peptides of SEQ ID NO 5 of ApoCIII

16. A vaccine immunogen according to claim 2 further comprising P30 between two peptides of SEQ ID NO 5 of ApoCIII

17. A vaccine immunogen according to claim 2 further comprising P2 between two peptides of SEQ ID NO 4 of ApoCIII

18. A vaccine immunogen according to claim 2 comprising P30 between two peptides of SEQ ID NO 4 of ApoCIII

19. A vaccine according to claim 2, further comprising a pharmaceutically acceptable excipient, and optionally an adjuvant.

20. An isolated antibody elicited by the peptides as claimed in claim 1.

21. An isolated antibody elicited by the vaccine immunogen of claim 2.

22. A monoclonal antibody that is specific for the peptides as claimed in claim 1.

23. A monoclonal antibody as claimed in claim 22 wherein the monoclonal antibody is capable of being produced from one of the following hybridomas deposited at ECACC under the accession numbers 01080123, 01080122, 01080121, 01080120, or 01080124.

24. A monoclonal antibody that is capable of competing with those monoclonal antibodies claimed in claim 24, for binding to human ApoCIII.

25. A peptide comprising the epitope described in SEQ ID NO: 3 or mimotope or fragment thereof.

26. A vaccine immunogen comprising a peptide as claimed in claim 25, conjugated to a carrier molecule.

27. A vaccine immunogen according to claim 26 in which the link between the carrier and the peptide is through the C-terminus of the peptide.

28. A vaccine immunogen according to claim 26 in which the link between the carrier and the peptide is through the N-terminus of the peptide.

29. A vaccine immunogen according to claim 26 in which the carrier is linked to two or more peptides.

30. A vaccine immunogen according to claim 26 in which the carrier is recombinantly fused between two peptides.

31. A vaccine immunogen according to claim 26, wherein the carrier comprises a T-helper epitope.

32. A vaccine immunogen according to claim 26, wherein the carrier molecule is tetanus toxoid, or a peptide of tetanus toxin.

33. A vaccine immunogen according to claim 26, in which the carrier molecule is P2.

34. A vaccine immunogen according to claim 26, in which the carrier molecule is P30.

35. A vaccine immunogen according to claim 34 further comprising n+1 peptides and n carriers, in which the peptides and carriers are alternately fused to each other, and in which n=1, 2, 3, 4, 5, 6 or 7.

36. A vaccine immunogen according to claim 35 in which all the peptides have the same sequence.

37. A vaccine immunogen according to claim 26 further comprising P2 fused between two peptides of SEQ ID NO 2 of ApoCIII

38. A vaccine immunogen according to claim 26 further comprising P30 (peptide of TT) between two peptides of SEQ ID NO 2 of ApoCIII

39. A vaccine immunogen according to claim 26 further comprising P2 between two peptides of SEQ ID NO 5 of ApoCIII.

40. A vaccine immunogen according to claim 26 further comprising P30 between two peptides of SEQ ID NO 5 of ApoCIII.

41. A vaccine immunogen according to claim 26 further comprising P2 between two peptides of SEQ ID NO 4 of ApoCIII.

42. A vaccine immunogen according to claim 26 further comprising P30 between two peptides of SEQ ID NO 4 of ApoCIII.

43. A vaccine according to claim 26 further comprising a pharmaceutically acceptable excipient, and optionally an adjuvant.

44. An isolated antibody elicited by the peptides as claimed in claim 25.

45. An isolated antibody elicited by the vaccine immunogen of claim 26.

46. A monoclonal antibody that is specific for the peptides as claimed in claim 25.

47. A monoclonal antibody as claimed in claim 46 wherein the monoclonal antibody is capable of being produced from one of the following hybridomas deposited at ECACC under the accession numbers 01080123, 01080122, 01080121, 01080120, or 01080124.

48. A monoclonal antibody that is capable of competing with those monoclonal antibodies claimed in claim 47, for binding to human ApoCIII.

49. A peptide comprising a epitope described in SEQ ID NO: 4-47 or mimotope or fragment thereof.

50. A vaccine immunogen comprising a peptide as claimed in claim 49, conjugated to a carrier molecule.

51. A vaccine immunogen according to claim 50 in which the link between the carrier and the peptide is through the C-terminus of the peptide.

52. A vaccine immunogen according to claim 50 in which the link between the carrier and the peptide is through the N-terminus of the peptide.

53. A vaccine immunogen according to claim 50 in which the carrier is linked to two or more peptides.

54. A vaccine immunogen according to claim 50 in which the carrier is recombinantly fused between two peptides.

55. A vaccine immunogen according to claim 50, wherein the carrier comprises a T-helper epitope.

56. A vaccine immunogen according to claim 50 wherein the carrier molecule is tetanus toxoid, or a peptide of tetanus toxin.

57. A vaccine immunogen according to claim 50, in which the carrier molecule is P2.

58. A vaccine immunogen according to claim 50, in which the carrier molecule is P30.

59. A vaccine immunogen according to claim 58 comprising n+1 peptides and n carriers, in which the peptides and carriers are alternately fused to each other, and in which n=1, 2, 3, 4, 5, 6 or 7.

60. A vaccine immunogen according to claim 59 in which all the peptides have the same sequence.

61. A vaccine immunogen according to claim 50 further comprising P2 fused between two peptides of SEQ ID NO 2 of ApoCIII

62. A vaccine immunogen according to claim 50 further comprising P30 (peptide of TT) between two peptides of SEQ ID NO 2 of ApoCIII

63. A vaccine immunogen according to claim 50 further comprising P2 between two peptides of SEQ ID NO 5 of ApoCIII

64. A vaccine immunogen according to claim 50 further comprising P30 between two peptides of SEQ ID NO 5 of ApoCIII

65. A vaccine immunogen according to claim 50 further comprising P2 between two peptides of SEQ ID NO 4 of ApoCIII

66. A vaccine immunogen according to claim 50 further comprising P30 between two peptides of SEQ ID NO 4 of ApoCIII

67. A vaccine according to claim 50 further comprising a pharmaceutically acceptable excipient, and optionally an adjuvant.

68. An isolated antibody elicited by the peptides as claimed in claim 49.

69. An isolated antibody elicited by the vaccine immunogen of claim 50.

70. A monoclonal antibody that is specific for the peptides as claimed in claim 49.

71. A monoclonal antibody as claimed in claim 70 wherein the monoclonal antibody is capable of being produced from one of the following hybridomas deposited at ECACC under the accession numbers 01080123, 01080122, 01080121, 01080120, or 01080124.

72. A monoclonal antibody that is capable of competing with those monoclonal antibodies claimed in claim 71, for binding to human ApoCIII.

73. A method of treatment or prophylaxis of atherosclerosis of an individual in need thereof, by administration of a vaccine immunogen or vaccine as claimed in any one of claim 2, 25, or 50 to said individual.

74. A method of treatment or prophylaxis of atherosclerosis of an individual in need thereof, by administration of a monoclonal antibody as claimed in any one of claim 27, 46, or 70 to said individual.

75. Use of a peptide as claimed in any one of claim 1, 25, or 49 in the manufacture of a medicament for the prevention or treatment of atherosclerosis.
